(19) United States
(12) Patent Application Publication
(10) Pub. No.: US 2013/0274336 A1
(43) Pub. Date: Oct. 17, 2013

(54) TRANS10:CIS12 ISOMER OF CONJUGATED LINOLEIC ACID AS A THERAPEUTIC AND PREVENTATIVE AGENT FOR HYPERTENSION SPECIFIC TO PREGNANCY

(71) Applicant: WISCONSIN ALUMNI RESEARCH FOUNDATION, Madison, WI (US)

(72) Inventors: Ian Michael Bird, Madison, WI (US); Manish Suresh Patankar, Fitchburg, WI (US); Derek Steven Boeldt, Fitchburg, WI (US); Mian Mohammed Khuram Shahzad, Madison, WI (US)

(73) Assignee: WISCONSIN ALUMNI RESEARCH FOUNDATION, Madison, WI (US)

(21) Appl. No.: 13/787,923

(22) Filed: Mar. 7, 2013

Related U.S. Application Data
(60) Provisional application No. 61/623,815, filed on Apr. 13, 2012.

(51) Int. Cl. A61K 31/201; A23C 9/152; A23L 1/30

(52) U.S. Cl. CPC A61K 31/201; A23L 1/3008; A23C 9/1528
USPC 514/560

(57) ABSTRACT

Described herein are compositions and methods for the use of t10:c12 conjugated linoleic acid to reduce hypertension in pregnant women. Advantageously, by reducing blood pressure, the compositions will also reduce the risk of pre-term birth. The t10:c12 conjugated linoleic acid compositions are advantageously food products, particularly dairy products. Also included are methods of determining the t10:c12 conjugated linoleic acid content of a dairy product and optionally enriching the dairy product for t10:c12 conjugated linoleic acid. Enriched dairy products can be labeled for use during pregnancy.

### CROSS-REFERENCE TO RELATED APPLICATIONS

This application claims priority to U.S. Provisional Application 61/623,815 filed on Apr. 13, 2012, which is incorporated herein by reference in its entirety

### STATEMENT REGARDING FEDERALLY SPONSORED RESEARCH & DEVELOPMENT

This invention was made with government support under HD069181 awarded by the National Institutes of Health. The government has certain rights in the invention.

### FIELD OF THE DISCLOSURE

The present disclosure is related to compositions and methods for reducing the risk of pre-term birth in pregnant women, particularly women at risk of preeclampsia.

### BACKGROUND

Pregnancy-induced hypertension (PIH) occurs in five to twenty percent of all human pregnancies. PIH-related disorders are a leading global cause of maternal and infant illness and death. PIH occurs in millions of births a year and is responsible for about 15 percent of all premature births. By conservative estimates, PIH-related disorders are responsible for 76,000 deaths each year. The risk of death for a pregnant woman with severe preeclampsia is 0.5%, and the risk of perinatal death for her baby is 13%; if the condition remains untreated and eclampsia develops, the risk of maternal and fetal death increases to 5% and 28%, respectively.

The term PIH includes both preeclampsia and gestational hypertension. Preeclampsia is a rapidly progressive condition, characterized by the occurrence of both high blood pressure and abnormal levels of protein in the urine (proteinuria). Eclampsia is in turn a more severe form of preeclampsia that is also characterized by seizures. Gestational hypertension is hypertension in pregnancy without proteinuria, and it may be a less severe form of preeclampsia or a precursor to preeclampsia. Preeclampsia and gestational hypertension may be further classified as mild or severe depending upon the severity of the clinical symptoms.

There is much evidence to support the case that pregnancy requires a general enhancement of vasodilation, and when this fails to occur, pregnancy induced hypertension (PIH) results. In general, the clinical symptoms of PIH occur in the late second trimester or in the third trimester of pregnancy, although symptoms may occur earlier in pregnancy. PIH may be superimposed over other forms of hypertension, such as essential and secondary hypertension, that exist prior to or develop early in pregnancy.

Clinically, PIH is a syndrome having maternal and fetal manifestations. The maternal condition, for example, is characterized by vasospasm, activation of the coagulation system, oxidative stress and inflammatory-like responses, all of which have detrimental effects on the placenta, kidney, blood, liver, vasculature, cardiopulmonary system and brain. PIH is a systemic syndrome, and several of its non-hypertensive symptoms and complications may be life-threatening even with only mild increases in blood pressure.

After a diagnosis of severe PIH, the baby is generally induced and delivered if it is near term, i.e., after 36 weeks. However, if PIH occurs earlier in the pregnancy, its impact is more serious because fetal viability is lower; infant death occurs in approximately 87% of these cases. For pregnancies in which PIH occurs earlier than 24 weeks, the induction of labor is recommended and results in essentially 100% neonatal mortality. For pregnancies between 24 and 28 weeks of gestation, management of PIH may be attempted to increase gestational age, provided that there is close monitoring for maternal and fetal complications.

Current treatments for PIH are aimed at direct inhibition of vasoconstriction using therapies targeting vascular smooth muscle contraction, but this approach does not address the cause of PIH. Indeed such therapies are also not always successful. What are needed are improved compositions and methods for the management of pregnancy-induced hypertension that address the cause, i.e., restore pregnancy enhanced vasodilation.

### BRIEF SUMMARY

In one aspect, a method of reducing the risk of pre-term birth associated with hypertension comprises identifying a woman diagnosed with pregnancy induced hypertension or at risk of preeclampsia, and administering to the woman an effective amount of a t10:c12 conjugated linoleic acid composition comprising t10:c12 conjugated linoleic acid or a pharmaceutically acceptable salt, triglyceride, or ester thereof.

In another aspect, a method of preparing a dairy product comprises

determining the amount of t10:c12 conjugated linoleic acid in the dairy product, and

a) if the amount of t10:c12 conjugated linoleic acid is greater than that sufficient to achieve a response to the t10:c12 conjugated linoleic acid in vivo, packaging the dairy product in a container identifying the dairy product as for use during pregnancy, or

b) if the amount of t10:c12 conjugated linoleic acid is less than that sufficient to achieve a response to the t10:c12 conjugated linoleic acid in vivo, enriching the dairy product with t10:c12 conjugated linoleic acid or a pharmaceutically acceptable salt, triglyceride, or ester thereof, to reach an the amount of t10:c12 conjugated linoleic acid greater than or equal to that sufficient to achieve a response to the t10:c12 conjugated linoleic acid in vivo, and packaging the dairy product in a container identifying the dairy product as for use during pregnancy.

In yet another aspect, a prenatal vitamin, comprises t10:c12 conjugated linoleic acid or a pharmaceutically acceptable salt, triglyceride or ester thereof, folic acid, and a pharmaceutically acceptable excipient.

### BRIEF DESCRIPTION OF THE DRAWINGS

FIG. 1 shows the structure of the active t10:c12 CLA isomer and the inactive c9:t11 CLA isomer.

FIG. 2 is a comparison of [Ca²⁺]i and NO responses in single endothelial cells while still on intact sheep uterine artery (left) or human umbilical vein (right).

FIG. 3 shows the effect of 30 minute pretreatment of 10 ng/ml equivalents of P1GF, VEGFE and VEGF165 (all 10 ng/ml), along with the rescue effect of U0126 (10 uM) or PP2 (10 uM) on VEGF165 mediated inhibition.*P<0.05.

FIG. 4 shows the effect of 30 minute pretreatment of 10 ng/ml VEGF165, along with the rescue effect of t10:c12 CLA (50 uM) or PP2 (10 uM) on VEGF165 mediated inhibition. *P<0.05

The above-described and other features will be appreciated and understood by those skilled in the art from the following detailed description, drawings, and appended claims.

### DETAILED DESCRIPTION

Described herein are methods of reducing hypertension by administering a t10:c12 conjugated linoleic acid composition. In one embodiment, the t10:c12 conjugated linoleic acid composition is administered to a woman diagnosed with pregnancy-induced hypertension or at risk of preeclampsia. The woman at risk of preeclampsia can be a woman who is pregnant or a woman who is planning to become pregnant. By reducing hypertension in a woman diagnosed with pregnancy-induced hypertension or at risk of preeclampsia, the risk of pre-term birth associated with hypertension in pregnant women will also be reduced. Also included herein are compositions containing the t10:c12 conjugated linoleic acid such as dairy products and prenatal vitamins suitable for administration during pregnancy.

### Definitions

As used herein, “conjugated linoleic acid” or “CLA” refers to a conjugated linoleic acid, that is, an unsaturated fatty acid having 18 carbons and two conjugated double bonds. This term encompasses all positional and geometric isomers of linoleic acid with two conjugated carbon-carbon double bonds in the molecule. Exemplary CLAs include 9c:11t; 9t:11c: 10c:12t and 10t:12c. CLA differs from ordinary linoleic acid in that ordinary linoleic acid has double bonds at carbon atoms 9 and 12. As used herein, “CLA” encompasses a single isomer, a selected mixture of two or more isomers, and a non-selected mixture of isomers obtained from natural sources, as well as synthetic and semisynthetic CLA.

Also included herein are salts of CLA, such as those formed by reacting the free acid with a pharmaceutically acceptable base. Further included are triglycerides and esters of CLA. As used herein, it should be understood that the term CLA also includes its salt, triglyceride and ester forms.

“Pharmaceutically acceptable salts” of CLA are formed by making an acid or base salt thereof, and further refers to pharmaceutically acceptable solvates of such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable.

As used herein, “c” refers to a chemical bond in the cis orientation, and “t” refers to a chemical bond in the trans orientation. If a positional isomer of CLA is designated without a “c” or a “t”, that designation includes all four possible isomers. For example, CLA encompasses c10:t12; t10:c12; t10:t12; and c10:c12 CLA, while c10:t12 CLA refers to just the single isomer.

As used herein, the term “conjugated linoleic acid composition” refers to a composition containing conjugated linoleic acid or a derivative of CLA including, but not limited to, pharmaceutical compositions such as pills, capsules and tablets; food products, and the like. A composition includes CLA and at least one additional component such as a pharmaceutically acceptable excipient, or a food product. As used herein, the term “food product” refers to a food suitable for consumption by humans. The “food product” may be a prepared and packaged food (e.g., milk, yogurt, or cheese). Food products include both prepare food products. “Prepared food product” means a pre-packaged food approved for human consumption.

An exemplary food product is a dietary supplement such as a powder, liquid concentrate or shake. Powders and concentrates can be added to a liquid such as water or milk for consumption. In addition to c10:t12 CLA, the dietary supplement can comprise additional components, particularly those that are beneficial during pregnancy such as vitamins, minerals, amino acids and protein sources.

Another exemplary food product is a dairy product, such as that produced from the milk of cows, sheep, goats and the like. The term “milk” refers to the normal secretion obtained from the mammary glands of mammals. Dairy products include milk, cheese, yogurt, sour cream, butter, and the like.

The fat found in cow's milk, for example, normally includes up to about 1 wt % of CLA, a fraction of which is t10:c12 CLA. The amount of CLA in milk can be increased by feeding the animal a special diet supplemented with vegetable oil containing linoleic acid. Another means for increasing the amount of CLA in milk is by grazing animals on fresh pasture, thus organic milk is often higher in CLA than milk produced by conventional farming methods.

As used herein, a dairy product, such as milk, can be enriched with t10:c12 conjugated linoleic acid, alone or as a mixture with other isoforms such as t9:c11, to increase the amount of t10:c12 conjugated linoleic acid in the dairy product. Enriching a dairy product with t10:c12 conjugated linoleic acid includes altering the diet of the animals used to produce the dairy product to increase the amount of t10:c12 conjugated linoleic acid, or adding substantially pure t10:c12 conjugated linoleic acid to the dairy product, during or after production. The t10:c12 conjugated linoleic acid can be added to a milk product, which is then converted into another dairy product, such as cheese, yogurt, sour cream, or butter.

As used herein, a prenatal vitamin is defined as a dietary supplement typically recommended for pregnant women and/or women planning on becoming pregnant. Prenatal vitamins include vitamins and minerals such as iron, and folic acid, for example. Prenatal vitamins are in the form of an oral pharmaceutical composition as described herein. A prenatal vitamin for daily use comprises, for example, 10 mg to 40 mg of t10:c12 conjugated linoleic acid or a pharmaceutically acceptable salt, triglyceride, or ester thereof.

In one embodiment, t10:c12 CLA is added in the form of Clarinol®, a natural source of CLA currently used to reduce fat and increase lean muscle mass.

Methods of Reducing Pre-Term Birth with t10:c12 CLA

Despite extensive research efforts, the etiology of preeclampsia is not well-understood. Vascular endothelial activation followed by vasospasm appears to be a central feature in the pathogenesis of preeclampsia. Theories of its cause include abnormal implantation and development of the placenta, oxidative stress, impaired endothelial prostanoid and nitric oxide homeostasis, genetic polymorphisms, abnormal circulating autoantibodies, and an abnormal maternal systematic inflammatory response.

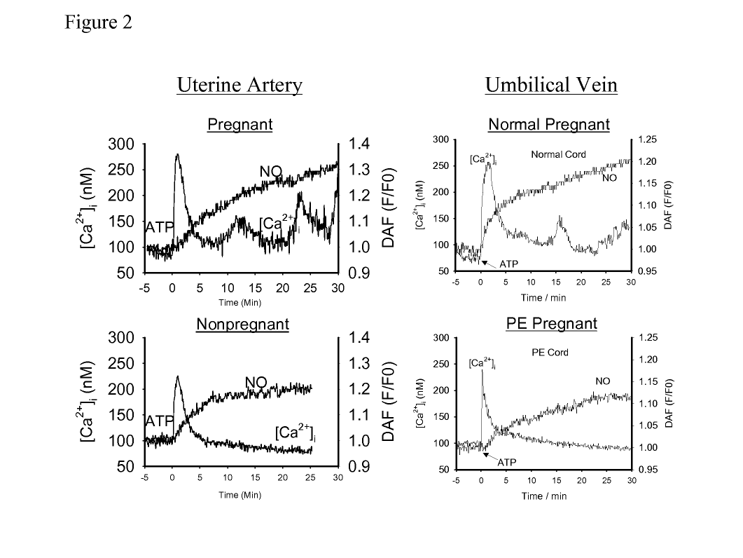

The molecular basis for vascular endothelial dysfunction has been even less well-understood. Recent studies of the endothelium in uterine artery and umbilical vein established that in normal pregnancy, elevated blood flow is dependent on the successful expression and coupling function of endothelial cell monolayers by intercellular gap junctions, and specifically the connexin 43 (CX43) type of gap junction. In a healthy pregnancy, endothelial cell coupling is high and associated with enhanced Ca²⁺ bursts, while in nonpregnancy they are low. There are also inflammatory and growth factor-related endocrine signals associated with a number of PIH conditions including preeclampsia that can disrupt this enhanced response and otherwise return the cells to a suboptimal state through kinase phosphorylation. It was previously shown that the preeclamptic umbilical vein endothelium shows a similar low level of gap junction activity with no repeated Ca²⁺ bursts, similar to non-pregnant endothelium (FIG. 2). Vascular endothelial growth factor (VEGF) appears to be at least one major effector of this inhibitory activity. The two kinase pathways that can achieve this inhibition include ERK and Src. It is demonstrated herein that the pharmacologic agents U0126 (a blocker of the ERK pathway) and PP2 (a blocker of the Src pathway) can rescue the cells from the inhibitory effects of VEGF on subsequent ATP stimulated Ca²⁺ bursts. It has also been confirmed this rescue is associated with a reversal of the phosphorylation of CX43 at specific inhibitory sites. However, while PP2 in particular can affect the rescue of uterine artery cells in vitro, it is not a selective drug, and being synthetic may also not be well-tolerated. It would be advantageous to identify natural products that have the same effects.

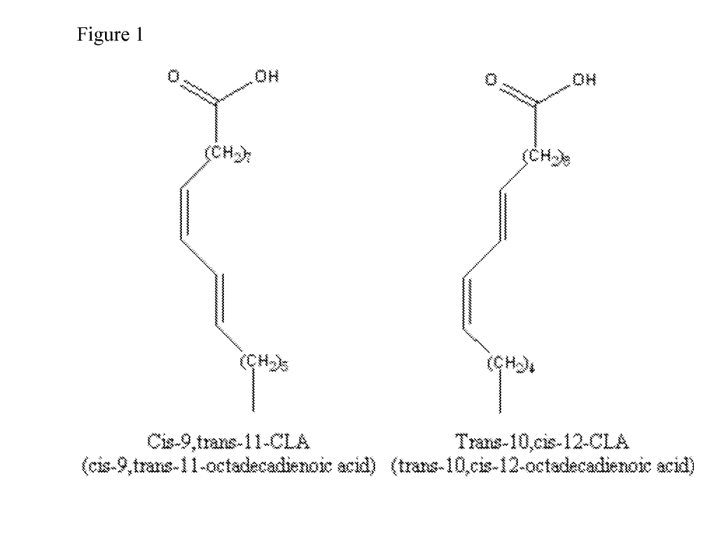

It has recently been shown that CLA, particularly the t10:c12 isomer, is an agent useful for inhibiting the growth of ovarian tumors. One of the hallmarks of the growth of cancer cells is the action of kinase signaling pathways, which can involve sustained ERK and Src signaling. It was shown that t10:c12 CLA can inhibit these pathways, leading to the hypothesis that t10:c12 CLA would also inhibit ERK and Src signaling in uterine artery cells, thus rescuing the cells from the inhibitory effects of VEGF on subsequent ATP stimulated Ca²⁺ bursts. In the patient with PIH, including preeclampsia or at risk of preeclampsia, administration of an effective amount of t10:c12 CLA is expected to restore/preserve vascular endothelial function. FIG. 1 shows the structure of the active t10:c12-CLA and the inactive isomer c9:t11 CLA. By restoring/preserving vascular endothelial function in the pregnant uterus, t10:c12-CLA will reduce hypertension in pregnant women and thus reduce the risk of pre-term birth.

In one embodiment, a method of reducing the risk of pre-term birth associated with hypertension comprises identifying a woman diagnosed with pregnancy-induced hypertension or at risk of preeclampsia and administering to the woman an effective amount of a t10:c12 conjugated linoleic acid composition. In general, the effective amount of t10:c12 conjugated linoleic acid administered is an amount sufficient to achieve a response to the t10:c12 conjugated linoleic acid. In one embodiment, an amount sufficient to achieve a response to the t10:c12 conjugated linoleic acid is an amount that provides a blood level of 0.1 μM to 50 μM in the woman's bloodstream. Because t10:c12 CLA is nontoxic and generally regarded as safe, higher amounts are possible. In certain embodiments, when taken orally, the t10:c12 conjugated linoleic acid is administered in an amount of 10 mg to 40 g per day, more specifically 0.1 g to 10 g per day. The method optionally comprises monitoring blood pressure in the woman, such as at least once per day, or more, or less frequently such as once per week.

As used herein, hypertension is defined as systolic blood pressure of over 140 mm Hg and/or a diastolic blood pressure of over 90 mm Hg. Pregnancy induced hypertension includes gestational hypertension and preeclampsia. Gestational hypertension is hypertension during pregnancy that is not accompanied by protein in the urine. In one embodiment, the woman has, in addition to hypertension, protein in the urine, which is a classical symptom of preeclampsia. Preeclampsia includes mild preeclampsia, sometimes simply referred to as preeclampsia, and severe preeclampsia. Mild preeclampsia is defined as a systolic blood pressure of over 140 mm Hg and/or a diastolic blood pressure of over 90 mmHg, and a 0.3 g or more of protein in a 24 hour urine collection. Severe preeclampsia is defined as a systolic blood pressure of over 160 mm Hg and/or a diastolic blood pressure of over 110 mmHg, and a 5 g or more of protein in a 24 hour urine collection. In general, preeclampsia is identified after 20 weeks of gestation.

The methods described herein are useful for women diagnosed with pregnancy induced hypertension as well as women at risk for preeclampsia. An increased risk for preeclampsia is associated with first time pregnancies, when there is a large interval between pregnancies, pregnant women under the age of 20 or over the age of 35, women of black race, multi-gestational pregnancies, women who have conceived through in vitro fertilization (“IVF”), women who have had a prior pregnancy with PIH, women who have had a prior pregnancy conceived with a different partner, women with a family history of PIH or high blood pressure or diabetes, women who are of higher than normal weight or body mass index prior to pregnancy, undernutrition, women with a personal history of polycystic ovarian syndrome, insulin resistance or diabetes, hypertension, renal (kidney) disease, rheumatoid arthritis, systemic lupus erythematosus or other autoimmune diseases, or thrombophilia risk factors. The risk of recurrent PIH in subsequent pregnancies is approximately thirty-three percent (33%), and PIH is superimposed in twenty-five percent (25%) of pregnancies in which chronic hypertension is present before pregnancy.

In the embodiment where the woman is at risk of preeclampsia is pregnant or planning to become pregnant, the t10:c12 CLA can be administered in the form of a prenatal vitamin.

In general, a safe and effective amount is that amount of t10:c12 CLA that, when ingested in purified form or as food supplement is about 10 mg to about 40 grams of t10:c12 CLA administered per day, specifically about 0.1 gram to about 10 grams per day. The amounts of CLA deemed therapeutically effective are those which result in a measurable decrease in blood pressure.

In embodiments wherein the woman has gestational hypertension or mild preeclampsia, the t10:c12 CLA is administered in an amount sufficient to elevate the amount of t10:c12 CLA in the woman's bloodstream to 0.1 μM to 10 μM. In this embodiment, the t10:c12 CLA can be administered as a prenatal vitamin, a t10:c12 CLA-enriched food product such as a t10:c12 CLA-enriched dairy product, or a nutritional supplement containing t10:c12 CLA.

In one embodiment, the woman diagnosed with pregnancy induced hypertension is further diagnosed with severe preeclampsia. In this embodiment, the t10:c12 conjugated linoleic acid composition can be an injectable composition or a suppository in order to quickly elevate the levels of t10:c12 conjugated linoleic acid in the woman's bloodstream to an amount of 10 μM, 50 μM or more.

Once the blood pressure of the woman with severe preeclampsia has decreased, for example to a systolic blood pressure of less than 160 mm Hg and/or a diastolic blood pressure of less than 110 mmHg, the amount of the t10:c12 CLA administered can be decreased, for example, by tapering over several days, until the level administered is that recommended for gestational hypertension or mild preeclampsia as indicated herein, such as an amount sufficient to elevate the amount of t10:c12 CLA in the woman's bloodstream to 0.1 to 10 μM. The decreased amount of t10:c12 CLA can be administered as a prenatal vitamin, a dairy product such as a t10:c12 CLA-enriched dairy product, or a nutritional supplement.

In one embodiment, particularly in the case of severe preeclampsia, the t10:c12 CLA is coadministered with a calcium channel blocker such as labetalol, nifedipine, diltiazem or verapamil. Calcium channel blockers have been used during pregnancy to treat high blood pressure and for the treatment of preterm labor.

In another embodiment, the t10:c12 CLA is coadministered with a corticosteroid such as betamethasone or dexamethasone. Corticosteroids cause the immature fetus's lungs to produce surfactants which lubricate the linings of the air sacs in the lungs and increase the chances of a premature infant to breathe on its own or with less respiratory treatment.

In one embodiment, administering to the woman a t10:c12 conjugated linoleic acid composition results in a delay in delivery date compared to the expected delivery date based on improvement of the woman's symptoms. When the symptoms of preeclampsia progress to the point of severe preeclampsia, a physician may decide to induce labor or perform a cesarean section. In general, pregnancies with preeclampsia complications are delivered prior to the 40 week date due to increased risks to the mother. Delivery before 37 weeks, however, is a problem for the baby because the baby may not be developed enough to survive alone, and may at least require extended treatment in a neonatal intensive care unit. The risk of early birth, i.e., pre-term birth, must be weighed against whether or not the baby's lungs are mature, and this can be achieved with glucocorticoids. Using CLA to control blood pressure and so delaying delivery by even one day can give the baby additional time to mature and also save on hospital costs.

### Pharmaceutical Compositions

In one embodiment, a t10:c12 conjugated linoleic acid composition is in the form of a pharmaceutical composition. When the t10:c12 conjugated linoleic acid is in the form of a pharmaceutical composition, the t10:c12 conjugated linoleic acid can be in the form of purified t10:c12 conjugated linoleic acid, that is a conjugated linoleic acid composition comprising greater than 98% t10:c12 conjugated linoleic acid. As used herein, “pharmaceutical composition” means therapeutically effective amounts of the compound together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions also include rectal compositions such as suppositories or retention enemas, e.g., containing conventional suppository bases such as cocoa butter or other glycerides.

Pharmaceutical compositions may conveniently be presented in unit dosage form and may be prepared by the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

In one embodiment, a pharmaceutical composition is a prenatal vitamin comprising folic acid and a pharmaceutically acceptable excipient.

### Enriched Dairy Products

Also included herein are methods of preparing dairy products for use in pregnant women, particularly women diagnosed with pregnancy induced hypertension or at risk of preeclampsia.

In one embodiment, a method of preparing a dairy product comprises

determining the amount of t10:c12 conjugated linoleic acid in the dairy product, and

a) if the amount of t10:c12 conjugated linoleic acid is greater than that sufficient to achieve a response to the t10:c12 conjugated linoleic acid in vivo, packaging the dairy product in a container identifying the dairy product as for use during pregnancy, or

b) if the amount of t10:c12 conjugated linoleic acid is less than that sufficient to achieve a response to the t10:c12 conjugated linoleic acid in in vivo, enriching the dairy product with t10:c12 conjugated linoleic acid or a pharmaceutically acceptable salt, triglyceride or ester thereof to reach an the amount of t10:c12 conjugated linoleic acid greater than that sufficient to achieve a response to the t10:c12 conjugated linoleic acid in vivo, and packaging the dairy product in a container identifying the dairy product as for use during pregnancy.

In one embodiment, the dairy product contains 0.05 mg t10:c12 conjugated linoleic acid per g of dairy product to 10 mg t10:c12 conjugated linoleic acid per g of dairy product, specifically 0.5 mg t10:c12 conjugated linoleic acid per g of dairy product to 10 mg t10:c12 conjugated linoleic acid per g of dairy product. Higher and lower amounts are contemplated so long as the amount is sufficient to achieve a response to the t10:c12 conjugated linoleic in vivo. While blood levels of 50 μM are expected to achieve a response in vivo, blood levels of 10 μM and even lower such as 1 μM or less may be sufficient.

For example, the container may be a milk carton, a sour cream container, a yogurt container, a butter container, and the like. In one embodiment, the dairy product is an organic dairy product such as organic milk.

In one embodiment, the container identifying the dairy product as for use during pregnancy has a visible symbol or statement, wherein the symbol or statement indicates that the dairy product is preferred for use during pregnancy. Preference for use or recommendation for use during pregnancy may be implied by the use of terms such as ‘licensed, approved, recommended’, while certification or guaranteed minimum content statements would be linked to a minimum content determined by assay.

The invention is further illustrated by the following non-limiting examples.

### EXAMPLE

### Example 1

### Effect of Pretreatment of Uterine Arterial (UA) Endothelial Cells with VEGF

Simultaneous [Ca²⁺]i and NO fluorescent imaging analysis was performed using a high-speed excitation and emission wavelength switching system as described in the art. After loading with both Diaminofluorescein-2 Diacetate (DAF-2 DA) (20 uM; Molecular Probes) and fura-2 AM (10 uM) for 90 min, the chamber with the isolated UA was mounted on an inverted microscope (Diaphot 150; Nikon) so that a ×20 phase/fluor objective was focused on the lumenal endothelium and individual endothelial cells were visualized The excitation light from a xenon lamp was filtered to provide wavelengths of 340 and 380 (for fura-2) and 480 nm (for DAF-2) with a high-speed wavelength switcher (Lambda 10-2; Sutter, Novato, Calif.). Emission light from endothelial cells was passed through a dichroic mirror (505 nm) and through an emission filter of 520 nm for both fura-2 and DAF-2 with a high-speed rotating filter wheel (Lambda 10-2; Sutter). The fluorescence images were recorded by a digital camera (PixelFly; Cooke). InCyt Im3 imaging and analysis software (Intracellular Imaging) was used to acquire, digitize, and store the images and data for off-line processing and statistical analysis. The relative fluorescence intensities for fura-2 and DAF-2 were quantitatively comparable. When the endothelium was only loaded with fura-2 AM, no signal at 480/535 nm (for NO imaging) was detected. In contrast, when the endothelium was only loaded with DAF-2 DA, no detectable signal could be recorded at 340/510- and 380/510-nm wavelengths (for [Ca²⁺]i imaging; data not shown). To reduce photobleaching of these fluorescent dyes, images were acquired at 5-s intervals. Background and autofluorescence were obtained from control-unloaded endothelium. F340/F380, a fluorescence ratio of excitation at 340 nm to that at 380 nm, was determined after background and autofluorescence subtraction, and [Ca²⁺]i was calculated in real time by comparison to a standard curve established for the same settings using buffers of known free [Ca²⁺]. Because there is no significant change in baseline fluorescence during a 60-min experiment, we expressed the intracellular NO production as relative fluorescence (f), which is the net increment of DAF-2 fluorescence relative to its basal value (f=F/F0), where F is DAF-2 fluorescence intensity obtained during experiments and F0 is its basal fluorescence intensity.

All treatments were undertaken in Krebs Buffer (125 mM NaCl, 5 mM KCl, 1 mM MgSO₄, 1 mM KH₂PO₄, 6 mM glucose, 2 mM CaCl₂, and 25 mM HEPES, pH 7.4) and treatment with ATP was with the sodium salt at 100 uM for 30 minutes, added at time zero. FIG. 2 shows a comparison of [Ca²⁺]i and NO responses in single endothelial cells while still on intact sheep uterine artery (left) or human umbilical vein (right). Cells on vessels were loaded with Fura-2 and DAF2 and challenged at time zero with 100 uM ATP. Note in single endothelial cells on the pregnant UA (top left) and normal pregnant UV (top right) we see repeated Ca²⁺ bursts and NO continues to rise for full 30 min. In cells on the nonpregnant UA (bottom left) and in the UV from preeclamptic (PE) pregnancy (bottom right) there are no repeated Ca²⁺ bursts, and NO plateaus at a lower level well before 30 min. Note in particular the similarity of the lack of bursts and associated blunting of NO output in nonpregnant derived uterine arteries and preeclamptic subject derived umbilical cord veins compared to normal pregnant controls in each case.

### Methods for Examples 2 and 3

Studies in ovine uterine artery endothelial cells from pregnant sheep (P-UAEC). P-UAEC are a well-established cell model of endothelial cells isolated from uterine arteries and maintained in culture to passage 3 before freezing for future use.

Isolation of UAECs: Uterine arteries were obtained from Polypay and mixed Western breed nonpregnant sheep (n=4) and pregnant ewes (n=6) at 120-130 days of gestation during nonsurvival surgery, as described in the art. Procedures for animal handling and protocols for experimental procedures were approved by the University of Wisconsin-Madison Research Animal Care Committees of both the School of Medicine and Public Health and the College of Agriculture and Life Sciences and follow the recommended American Veterinary Medicine Association guidelines for humane treatment and euthanasia of laboratory farm animals.

Fura-2 [Ca²⁺]i Imaging Studies: Uterine artery endothelial cells plated to 35-mm dishes with glass coverslip windows (MatTek Corp.) were grown to the required density (20%-100%, as described) and then loaded with 5 μM Fura-2 AM in Krebs buffer (125 mM NaCl, 5 mM KCl, 1 mM MgSO₄, 1 mM KH₂PO₄, 6 mM glucose, 2 mM CaCl₂, and 25 mM HEPES, pH 7.4) for 45 min. After rinsing and incubating a further 30 min to complete ester hydrolysis, Fura-2 loading was verified by viewing at 380-nm ultraviolet excitation on a Nikon inverted microscope (TS100; Melville, N.Y.). About 60 cells were then preselected and video recordings commenced using alternate excitation at 340 and 380 nm at 1-sec intervals and measuring emitted light using a digital camera. From the ratio of emission at 510 nm detected at the two excitation wavelengths, and by comparison to a standard curve established for the same settings using buffers of known free Ca²⁺ concentration, the [Ca²⁺]i was then calculated for individual cells in real time using InCyt Im2 software (Intracellular Imaging Inc., Cincinnati, Ohio).

Data in graphs is calculated comparing the cells repeated burst patterns before and after repeat treatment. ATP 100 μM 30 min followed by ATP 100 uM 30 min gives a marginal decline in number of Ca²⁺ bursts (as defined in the art). However other agents activating protein kinases such as ERK and Src cause closure of CX43 and so loss of associated bursts. Therefore to perform the analysis, ATP stimulation is performed once to establish an uninhibited response for each cell. Then the inhibitory treatment under study is given (VEGF, 10 ng/ml or equivalent). After 30 minutes the second ATP treatment begins and burst response is expressed as % of the first ATP response. To then further test the protective or rescue effect of kinase inhibitors we follow the same protocol but add the rescue agent right before the VEGF. U0126 is an MEK/ERK pathway blocked, PP2 is a Src pathway blocker, and CLA is the test compound. CLA action is most comparable to that of PP2 or indeed better.

### Example 2

### Inhibitory Effects of Factors on Subsequent ATP Burst Number in P-UAEC

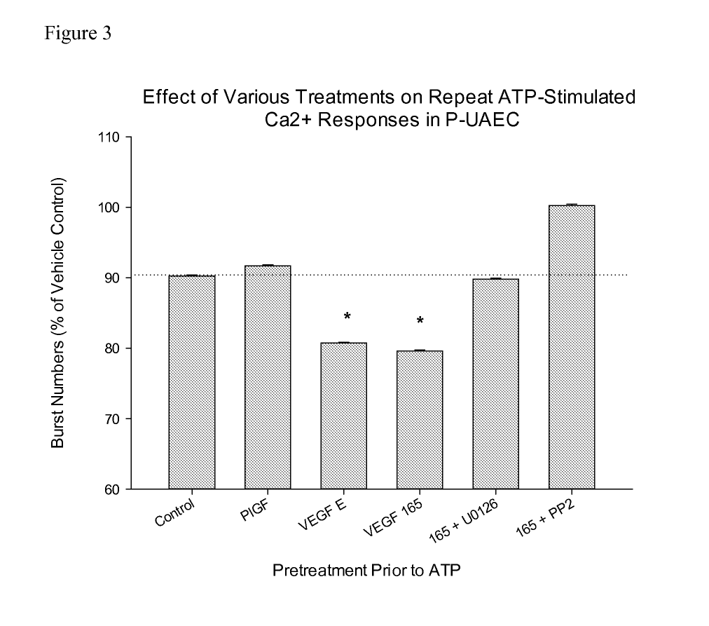

In cultured uterine artery endothelial cells from pregnant animals, the pharmacologic agents U0126 (a blocker of the ERK pathway) or PP2 (a blocker of the Src pathway) (FIG. 3) can rescue the cells from the inhibitory effects of VEGF on subsequent ATP stimulated Ca²⁺ bursts. It was also confirmed that this rescue is associated with a reversal of the phosphorylation of CX43 at specific inhibitory sites. FIG. 3 shows the effect of 30 min pretreatment of 10 ng/ml equivalents of P1GF, VEGFE and VEGF165 (all 10 ng/ml), along with the rescue effect of U0126 (10 μM) or PP2 (10 μM) on VEGF 165 mediated inhibition. *P<0.05.

### Example 3

### CLA Rescue of VEGF Inhibitory Effects on Subsequent ATP Burst Number in P-UAEC

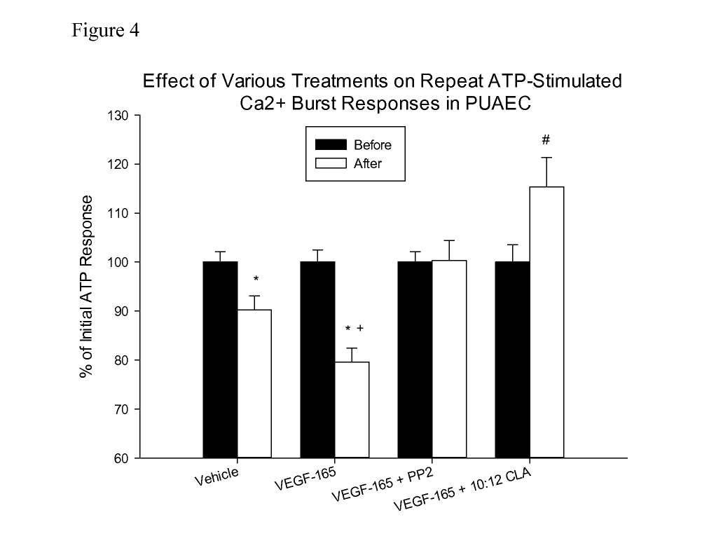

A more specific question is does t10:c12 CLA do the same thing in endothelium as in ovarian cancer cells, i.e., selectively block the ERK and Src pathways? If so, t10:c12 CLA is a better candidate for human therapy than PP2 because it is a natural product. As shown in FIG. 4, t10:c12 CLA can achieve the same rescue effect as PP2 in VEGF inhibited cells. Also that the effect of CLA actually achieves improvement of function beyond rescue. The effect is also isoform specific, since to date the runs with the c9:t11 CLA have not achieved rescue of the cells from inhibition.

Disclosed herein is a new use of t10:c 12 CLA to reduce hypertension in pregnant women such as women at risk of preeclampsia. Advantageously, the t10:c12 CLA can be administered as a food product such as a dairy product.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of reducing the risk of pre-term birth associated with hypertension, comprising
identifying a woman diagnosed with pregnancy induced hypertension or at risk of preeclampsia, and
administering to the woman an effective amount of a t10:c12 conjugated linoleic acid composition comprising t10:c12 conjugated linoleic acid or a pharmaceutically acceptable salt, triglyceride, or ester thereof.

2. The method of claim 1, wherein the t10:c12 conjugated linoleic acid is administered in an amount of 10 mg to 40 g per day.

3. The method of claim 1, further comprising monitoring blood pressure in the woman.

4. The method of claim 1, wherein administering to the woman a t10:c12 conjugated linoleic acid composition results in a delay in delivery date compared to the expected delivery date based on the woman's symptoms.

5. The method of claim 1, wherein the woman is diagnosed with gestational hypertension or mild preeclampsia.

6. The method of claim 5, wherein the t10:c12 conjugated linoleic acid composition is a food product or a nutritional supplement that has been enriched with t10:c12 conjugated linoleic acid.

7. The method of claim 6, wherein the food product is a dairy product that has been enriched with t10:c12 conjugated linoleic acid.

8. The method of claim 5, wherein the t10:c12 conjugated linoleic acid is administered in an amount sufficient to elevate the amount of t10:c12 conjugated linoleic acid in the woman's bloodstream to 0.1 μM to 10 μM.

9. The method of claim 1, wherein the woman at risk of preeclampsia is pregnant or planning to become pregnant.

10. The method of claim 9, wherein the woman at risk of preeclampsia is at 20 weeks or more of gestation.

11. The method of claim 9, wherein the t10:c12 conjugated linoleic acid composition is a prenatal vitamin.

12. The method of claim 1, wherein the woman diagnosed with pregnancy induced hypertension is diagnosed with severe preeclampsia, and the t10:c12 conjugated linoleic acid composition is an injectable or rectal pharmaceutical composition.

13. The method of claim 12, wherein the t10:c12 conjugated linoleic acid is administered in an amount sufficient to elevate the amount of t10:c12 CLA in the woman's bloodstream to more than 10 μM.

14. The method of claim 13, wherein when the woman's blood pressure has decreased to a systolic blood pressure of less than 160 mm Hg and/or a diastolic blood pressure of less than 110 mmHg, the dose of the t10:c12 conjugated linoleic acid is tapered to an amount sufficient to elevate the amount of t10:c12 CLA in the woman's bloodstream to 0.1 μM to 10 μM.

15. The method of claim 12, further comprising administering a calcium channel blocker.

16. A method of preparing a dairy product comprising
determining the amount of t10:c12 conjugated linoleic acid in the dairy product, and
a) if the amount of t10:c12 conjugated linoleic acid is greater than that sufficient to achieve a response to the t10:c12 conjugated linoleic acid in vivo, packaging the dairy product in a container identifying the dairy product as for use during pregnancy, or
b) if the amount of t10:c12 conjugated linoleic acid is less than that sufficient to achieve a response to the t10:c12 conjugated linoleic acid in vivo, enriching the dairy product with t10:c12 conjugated linoleic acid or a pharmaceutically acceptable salt, triglyceride, or ester thereof to reach an the amount of t10:c12 conjugated linoleic acid greater than that sufficient to achieve a response to the t10:c12 conjugated linoleic acid in vivo, and packaging the dairy product in a container identifying the dairy product as for use during pregnancy.

17. The method of claim 16, wherein the container identifying the dairy product as for use during pregnancy has a visible symbol or statement, wherein the symbol or statement indicates that the dairy product is preferred for use during pregnancy.

18. The method of claim 16, wherein the dairy product is an organic milk product.

19. A prenatal vitamin, comprising
t10:c12 conjugated linoleic acid or a pharmaceutically acceptable salt, triglyceride or ester thereof,
folic acid, and
a pharmaceutically acceptable excipient.

20. The prenatal vitamin of claim 19, comprising 10 mg to 40 mg of the t10:c12 conjugated linoleic acid or pharmaceutically acceptable salt, triglyceride, or ester thereof.
